(19) United States
(12) Patent Application Publication
(10) Pub. No.: US 2009/0317345 A1
(43) Pub. Date: Dec. 24, 2009

(54) Antiperspirant/Deodorant Gel Composition With Low Pouring Temperature

(75) Inventors: Vijay K. Joshi, Livingston, NJ (US); Heng Cai, Skillman, NJ (US); Christie M. Popoff, Morganville, NJ (US)

Correspondence Address: Joy S. Goudie;REVLON CONSUMER PRODUCTS CORPORATION, 237 PARK AVENUE, NEW YORK, NY, 10017, US

(73) Assignee: Revlon Consumer Products Corporation, New York, NY (US)

(21) Appl. No.: 12/478,843

(22) Filed: Jun. 5, 2009

Related U.S. Application Data
(60) Provisional application No. 61/075,247, filed on Jun. 24, 2008.

(51) Int. Cl. A61K 8/42; A61Q 15/00

(52) U.S. Cl. 424/65

(57) ABSTRACT

Solid stick antiperspirant and deodorant compositions containing both branched and linear N-acyl gelling agents along with dipropylene glycol are provided. Such compositions exhibit advantageous pouring temperatures below about 80° C.

This application claims priority from copending provisional application Ser. No. 61/075,247, filed Jun. 24, 2008, the entire disclosure of which is hereby incorporated by reference.

Cosmetic antiperspirant and deodorant products are often sold in the form of soft solids and sticks. The stick form, either in the shape of a rod or bar, has been popular in North America since it was introduced in the late 1970's, mainly because of its ease of application. Such products should be of sufficient firmness that a reasonable amount of active ingredient may be applied to the surface upon which it is applied.

Gelled compositions in soft solid or stick form are known and have been used for various cosmetic and pharmaceutical applications. Several N-acyl derivatives of amino acids, such as esters, amides, and amine salts, have been used as gellants with variable efficacy. One such N-acyl substituted amino acid amide gellant is N-lauroylglutamic acid di-n-butylamide (CTFA INCI name dibutyl lauroyl glutamide), commercially available from Ajinomoto Co., Inc, under the trade name GP-1. GP-1 has a linear N-acyl substituent. Another group of N-acyl substituted amino acid amide gellants, which includes N-2-ethylhexanoyl-L-glutamic acid dibutylamide (CTFA INCI name dibutyl ethylhexanol glutamide), commercially available from Ajinomoto Co., Inc. under the trade name EB-21. EB-21 has a branched N-acyl substituent.

The known art requires mixtures of gellants and carrier oils to be heated well above the gelling temperature before the gellant dissolves. It is recommended, however, that in case of anhydrous solid gel cosmetic compositions, the processing temperature should not be greater than 80° C. Higher temperatures tend to decompose fragrances and other components of cosmetic compositions producing, for example, malodors offensive to consumers.

Moreover, it is also impractical to re-dissolve a gellant having a high gelling temperature by re-heating such a composition once it has gelled. An elevated gelling temperature introduces a substantial risk that the composition would be gelled before it has been cooled to a temperature at which a temperature sensitive constituent can be introduced. There is also a risk that introduction of an active ingredient would rapidly lower the temperature below the oil gelling temperature, rendering subsequent handling extremely difficult on a bulk scale, such as filling of product dispensers.

Accordingly, it would be highly desirable if disadvantageous properties of gellants as mentioned above, could be reduced or eliminated. This would allow such gellants to be used effectively in cosmetic compositions such as antiperspirants and deodorants.

Another problem in the known art of gelling technology is a limitation in formulations. The carrier oils for solid gel compositions are limited to water immiscible hydrophobic materials. The formulations disclosed here are not limited.

The present invention relates to the surprising discovery that the addition of a polar solvent, ie dipropylene glycol to N-acyl amino acid derivative gellants and carrier oil compositions lowers the gelling temperature of the gellants, thereby lowering the pouring temperature of solid gel compositions to about 80° C. or below. For example, the addition of dipropylene glycol can lower the gelling temperature of a mixture of N-acyl amino acid amide derivative gellants including a branched N-acyl substituent, such as EB-21, and a linear N-acyl substituent, such as GP-1, to about 80° C. or below. Solid gel compositions having low pouring temperature are very useful for cosmetics compositions including, but not limited to, antiperspirants and deodorants.

Accordingly, one embodiment of the present invention provides solid gel cosmetic compositions comprising a mixture of N-acyl amino acid amide derivative gellants and dipropylene glycol wherein the pour temperature of the composition is about 80° C. or below.

Another embodiment the present invention provides solid gel cosmetic compositions comprising a mixture of branched and linear N-acyl substituted N-acyl amino acid amide derivative gellants and dipropylene glycol wherein the pour temperature of the composition is about 80° C. or below.

A further embodiment of the present invention provides solid gel cosmetic compositions comprising N-acyl amino acid amide derivative gellants EB-21 and GP-1, and dipropylene glycol wherein the pour temperature of the composition is about 80° C. or below.

Another embodiment the present invention provides solid gel cosmetic compositions comprising N-acyl amino acid amide derivative gellants EB-21 and GP-1, dipropylene glycol, and antiperspirant and/or deodorant active agents wherein the pour temperature of the composition is about 80° C. or below.

Still another embodiment of the present invention provides solid deodorant and antiperspirant commercial products which include EB-21 and GP-1 gellants and dipropylene glycol wherein the pour temperature of the product is about 80° C. or below.

The present invention relates to the surprising discovery that the addition of dipropylene glycol to N-acyl amino acid derivative gellants and carrier oil compositions lowers the gelling temperature of the gellants, thereby lowering the pouring temperature of solid gel compositions to less than about 80° C.

As used herein, the term “dissolution temperature” means the temperature at which the gellant or gellants are completely dissolved in the liquid phase.

As used herein, the term “gelling temperature” means the temperature at which a composition is transformed from liquid to gel, when the composition is heated until all ingredients dissolve and the composition in the liquid state is then cooled to form a solid gel.

Solid gel composition is defined by gel strength as measured using Texture Analyzer instrument TAXT2i and a compression probe TA-8B (Texture Technologies Corp. 18 Fairview Road Scarsdale, N.Y. 10583). The gel strength so measured should be from 50 g to 3000 gm, preferably from 150 gm to 2500 gm and more preferably from 350 gm to 2000 gm.

As used herein, the term “pouring temperature” means the minimum temperature at which a composition, when heated into the liquid state, can be poured into the desired form without gelling prematurely.

As used herein, the term “gellant” includes, but is not limited to, agents which form a semicrystalline structure by reaction with another material or by lowering of the temperature thereof while dissolved or colloidally suspended in a liquid medium. Gellants may form an extensive semicrystalline structure having interstices wherein a liquid in which the gellant was dissolved or suspended becomes encapsulated, hence forming a gel which comprises the semicrystallized gellant and the entrapped liquid.

As used herein, the term “N-acyl amino acid derivative gellants” include, but are not limited to, N-acyl substituted amino acid derivatives, such as esters, amides, and amine salts, described, for example, in U.S. Pat. No. 7,347,991 B2 and U.S. Pat. No. 7,347,992 B2, the descriptions are hereby incorporated by reference.

As used herein, the term “EB-21” means N-2-ethylhexanoyl-L-glutamic acid dibutylamide (CTFA INCI name dibutyl ethylhexanoyl glutamide), which is available from Ajinomoto Co., Inc. under the trade name EB-21.

As used herein, the term “GP-1” means N-lauroylglutamic acid di-n-butylamide (CTFA INCI name dibutyl lauroyl glutamide), which is available from Ajinomoto Co., Inc. under the trade name GP-1.

As used herein, the term “dipropylene glycol” means the organic compound with the structure formula of CH₃—CHOH—CH₂—O—CH₂—CHOH—CH₃, also known as oxybispropanol, di-sec-alcohol, or bis(2-hydroxy-propyl)ether. The term “dipropylene glycol” includes, but is not limited to, the isomer mixture of 1,1′-Oxybis(2-propanol), 2-(2-Hydroxypropoxy)-1-propanol, and 2,2′-Oxybis(1-propanol), or any one or two of the isomers therein.

As used herein, the term “at least one” means one or more of the item to which it makes reference.

As used herein, “about” shall generally mean within 20 percent, preferably within 10 percent, and more preferably within 5 percent of a given value or range.

One embodiment the present invention provides solid gel cosmetic compositions comprising a mixture of N-acyl amino acid amide derivative gellants and dipropylene glycol. Another embodiment the present invention provides solid gel cosmetic compositions comprising a mixture of N-acyl amino acid amide derivative gellants with one or more branched N-acyl substituents and one or more linear N-acyl substituents, and dipropylene glycol. Another embodiment the present invention provides solid gel cosmetic compositions comprising N-acyl amino acid amide derivative gellants EB-21 and GP-1, and dipropylene glycol. In a another embodiment the present invention provides solid gel cosmetic compositions comprising N-acyl amino acid amide derivative gellants ES-21 and GP-1, dipropylene glycol, and antiperspirant and/or deodorant active agents. Still another embodiment of the present invention provides solid deodorant and antiperspirant commercial products which include EB-21 and GP-1 gellants and dipropylene glycol.

The above described embodiments may have pour temperatures of about 80° C. or below. In some embodiments the pour temperatures are below about 75° C.; in other embodiments the pour temperatures are below about 70° C., about 65° C., about 60° C., about 55° C. and about 50° C.

Cosmetic antiperspirant and deodorant compositions typically comprise an antiperspirant active that is dissolved or suspended in a cosmetically acceptable carrier material comprising one or a mixture of cosmetically acceptable water-immiscible oils. Gelled antiperspirant and/or deodorant compositions in soft solid or stick form may comprise gellants, dipropylene glycol, carrier oils, antiperspirant and/or deodorant actives, and other optional ingredients.

Cosmetic compositions typically comprise at least one optional ingredient that is dissolved or suspended in a cosmetically acceptable carrier material comprising one or more of a mixture of cosmetically acceptable water-immiscible oils in soft solid or stick form may comprise gellants, dipropylene glycol, carrier oils, and other optional ingredients.

Gellants

The present invention relates in part to cosmetic compositions in which a water-immiscible oil phase is solidified using one or more N-acyl substituted amino acid derivative gellants. N-acyl substituted amino acid derivatives, such as esters, amides, and amine salts, are described, for example, in U.S. Pat. No. 7,347,991 B2 and U.S. Pat. No. 7,347,992 B2, incorporated herein by reference.

N-acyl substituted L-glutamic acid or L-aspartic acid derivatives are preferred. Such gellants can be represented by this general formula:

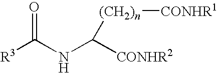

wherein R¹ and R² independently represents a linear or branched alkyl group having 1 to 26 carbon atoms; R³ represents a linear or branched alkyl group having 7 to 10 carbon atoms; n represents 1 or 2 provided that the acidic amino acid residue in the molecule is L-aspartic acid residue when n is 1 and said amino acid residue is L-glutamic acid residue when n is 2.

The linear N-acyl substituted N-lauroylglutamic acid di-n-butylamide (CTFA INCI name dibutyl lauroyl glutamide) and the branched N-acyl substituted N-2-ethylhexanoyl-L-glutamic acid dibutylamide (CTFA INCI name dibutyl ethylhexanol glutamide) are available commercially from Ajinomoto Co. Ltd. under the trade names GP-1 and EB-21, respectively.

Additional N-acyl substituted amino acid derivative gellants are described in the specification of U.S. Pat. No. 3,969,087 description herein incorporated by reference and U.S. Pat. No. 7,347,991 B2 and may be used along with or in place of GP-1 and EB-21 in certain embodiments, particularly where combinations of linear and branched N-acyl substituents are used together along with dipropylene glycol.

An ester or amide of an N-acyl amino acid may be obtained by reacting an N-acyl amino acid with an alcohol or an amine (inclusive of ammonia) in the presence or absence of an acidic catalyst while heating, The amine salt of the N-acyl amino acid may be obtained by neutralizing the N-acyl amino acid with an amine.

N-acyl substituted L-glutamic acid or L-aspartic acid derivatives may be produced by, for example, reacting a long chain fatty acid halide with L-glutamic acid or L-aspartic acid in the presence of a basic catalyst according to the Schotten Baumann's reaction to prepare an N-acylated glutamic acid or N-acylated aspartic acid, and then reacting the resulting product with an amine derivative such as alkylamines in the presence of an acid catalyst or in the absence of a catalyst with heating. Alternatively, the target compound may be produced by reacting glutamic acid or an aspartic acid with an amine derivative such as alkylamines in the presence of an acid catalyst or in the absence of a catalyst, and then subjecting the resulting glutamic acid amide or aspartic acid amide to N-acylation by using an acylating agent such as aliphatic acid halides.

The weight proportion of N-acyl substituted amino acid gellants in the composition is commonly selected in the range of about 0.2 to about 20% and is preferably between about 1% to about 8% for the formation hard oil gels. The weight proportion of the gellant in the composition or water-immiscible phase is selected in concert with the choice of co-gellant or gellants, the weight of co-gellant or gellants and the desired hardness of the stick.

Dipropylene Glycol

Concentrations of dipropylene glycol may be between about 0.1% and about 40% by weight of the final composition. In certain embodiments, concentrations of the dipropylene glycol may be between about 2% and about 32% by weight of the final composition. In other embodiments, concentrations of dipropylene glycol may be between about 2% and about 20% by weight of the final composition. In further embodiments, concentrations of the dipropylene glycol may be about 10% by weight of the final composition.

Carrier Liquids

Water-immiscible carrier liquids for the continuous phase may comprise one or a mixtures of materials which are relatively hydrophobic so as to be immiscible in water. Following partition between the continuous phase and the disperse phase, a small fraction of hydrophilic liquid may remain in the continuous phase, provided the overall carrier liquid mixture is immiscible with water. It will generally be desired that the carrier oils mixture is liquid (in the absence of structurants) at temperatures of 15° C. and above. It may have some volatility but its vapor pressure will generally be less than 4 kPa (30 mmHg) at 25° C. so that the material can be referred to as an oil or mixture of oils. More specifically, it is generally desirable that at least about 80% by weight of the hydrophobic carrier liquid should consist of materials with a vapor pressure no higher than 4 kPa at 25° C.

In certain embodiments, a hydrophobic carrier material may include a volatile liquid silicone, i.e. liquid polyorganosiloxane, to provide a “drier” feel to the applied film after the composition is applied to skin. Such “volatile” material typically has a measurable vapor pressure at 20° C. or 25° C. Typically the vapor pressure of a volatile silicone lies in a range from 1 or 10 Pa to 2 kPa at 25° C.

Volatile polyorganosiloxanes may be linear or cyclic or mixtures thereof. Cyclic siloxanes include polydimethylsiloxanes, particularly those containing from 3 to 9 silicon atoms. In some embodiments, polydimethylsiloxanes contain no more than 7 silicon atoms an in other embodiments from 4 to 6 silicon atoms. Representative polyorganosiloxanes include cyclomethicones, such as cyclotetrasiloxane, cyclopentasiloxane and cyclohexasiloxane. Linear siloxanes also include polydimethylsiloxanes containing from 3 to 9 silicon atoms. The volatile silicones may also comprise branched linear or cyclic siloxanes such as the aforementioned linear or cyclic siloxanes substituted by one or more pendant —O—Si(CH₃)₃ groups. The volatile siloxanes by themselves usually exhibit viscosities below 10⁻⁵ m²/sec (10 centistokes), and particularly above 10⁻⁷ m²/sec (0.1 centistokes). The linear siloxanes usually exhibit vicosities below 5×10⁻⁶ m²/sec (5 centistokes). Examples of commercially available silicone oils include oils having grade designations 244, 245, 246, 344, and 345 from Dow Corning Corporation; other examples include Silicone 7158 and Silicone 7207 from Union Carbide Corporation; and SF1202 from General Electric.

Hydrophobic carriers employed in compositions according to the present invention may alternatively or additionally comprise non-volatile silicone oils, which include polyalkyl siloxanes, polyalkylaryl siloxanes and polyethersiloxane copolymers. These may be selected from, for example, dimethicone and dimethicone copolyols. Commercially available non-volatile silicone oils include products available under the trademarks Dow Corning 200 series, Dow Corning 556, and Dow Corning 704 (DC704).

Water-immiscible liquid carriers may contain from 0% to 100% by weight of one or more liquid silicones. In certain embodiments, liquid silicone may be 10-15% by weight of the whole composition.

Silicon-free hydrophobic carrier liquids may be used instead of, or in addition to, liquid silicones. Silicon-free hydrophobic organic liquids may include liquid aliphatic hydrocarbons such as mineral oils or hydrogenated polyisobutene, often selected to exhibit a low viscosity. Further examples of liquid hydrocarbons include polydecene, paraffins and isoparaffins of at least 10 carbon atoms.

Other suitable hydrophobic carriers liquids comprise liquid aliphatic or aromatic esters. Suitable aliphatic esters contain at least one long chain alkyl group, such as esters derived from C₁ to C₂₀ alkanols esterified with a C₈ to C₂₂ alkanoic acid or C₆ to C₁₀ alkanedioic acid. The alkanol and acid moieties or mixtures thereof are preferably selected such that they each have a melting point of below 20° C. These esters include isopropyl myristate, lauryl myristate, isopropyl palmitate, diisopropyl sebacate and diisopropyl adipate. Suitable liquid aromatic esters, preferably have a melting point of below 20° C. and include fatty alkyl benzoates. Examples of such esters include suitable C₈ to C₁₈ alkyl benzoates or mixtures thereof such as C₁₂ to C₁₅ alkyl benzoates, e.g. those available under the trademark Finsolv. Aryl benzoate esters, such as benzyl benzoate ester also may be used.

Further examples of suitable hydrophobic carriers liquids include liquid aliphatic ethers derived from at least one fatty alcohol, such as myristyl ether derivatives, e.g. PPG-3 myristyl ether or lower alkyl ethers of polyglycols, such as an ether having named as PPG-14 butyl ether by the CTFA.

Aliphatic alcohols which are liquid at 20° C. also may be employed as carrier liquids in the present invention. It is usually desirable to employ aliphatic alcohols which are water-immiscible, and particular those having a boiling point of higher than 100° C. These include branched chain alcohols of at least 10 carbon atoms and in many instances up to 30 carbon atoms, particularly 15 to 25, such as isostearyl alcohol, hexyldecanol, octyldodecanol, and decyldecanol. Other suitable water-immiscible alcohols include benzyl alcohol and intermediate chain length linear alcohols, commonly containing from 9 to 13 carbon atoms, such as decanol or dodecanol. Such alcohols can also assist in the dissolution process of N-acyl substituted amino acid gellants. Such alcohols may constitute from at least 10% or 15% by weight of the water-immiscible liquid carrier mixture, and in certain embodiments may comprise up to 70% or 80% of the mixture. In certain formulations, the proportion of such aliphatic alcohols in said mixture may be from 10 or 15% to 30% by weight. In other formulations, the proportion may be greater than 30% by weight. However, aliphatic alcohols which are solid at 20° C., normally linear alcohols, such as stearyl alcohol are generally absent or present in no more than 3% by weight of the whole composition since they may lead to visible white deposits when a composition is topically applied to skin.

In some embodiments silicon-free liquids may constitute from 0-100% of the water-immiscible liquid carrier, although it is usually preferred that some silicone oil is present. The amount of silicon-free constituents may be up to 50% or 60% or even up to 80% of water-immiscible carrier liquid and in many instances from 10 to 60% by weight, e.g. 15 to 30% or 30 to 60% by weight, of the carrier liquid.

The compositions described herein may contain a more polar liquid, but usually only to the extent that it is miscible with the water-immiscible oil/mixture. In many instances this limits the proportion of such a constituent to about 15% w/w or less of the combined liquids, and in many instances to about 10% w/w or less on the same basis, though the amount may vary depending the compositions described herein may contain other low molecular weight polyhydric alcohols and oligomers thereof, commonly up to a molecular weight of about 150. This class may contain two hydroxyl substituents, as in ethylene glycol, propylene glycol, or a dihydroxyhexane or three hydroxy substituent as in glycerol.

Antiperspirant Active

Compositions described herein may contain an antiperspirant active ingredient. Antiperspirant actives may be incorporated in amounts from about 0.5-50%, or from about 5 to 30% or 40%, or from 5 or 10% to 30% of the weight of the composition. Generally speaking, the ability of a composition to control perspiration is directly proportional to the amount of the active antiperspirant ingredient included in the composition.

Antiperspirant actives for use herein are often selected from astringent active salts, including aluminum, and mixed aluminum/zirconium salts, including both inorganic salts, salts with organic anions and complexes. Astringent salts include aluminum, and aluminum/zirconium halides and halohydrate salts, such as chlorohydrates.

Antiperspirant activities of aluminum halohydrates (with Al:Chloride ratio of 1.2 to 2.2) are usually defined by the general formula Al₂(OH)ₓQ_{y}.w H₂O in which Q represents chlorine, bromine or iodine, x is variable from 2 to 5 and x+y=6 while w H₂O represents a variable amount of hydration. Especially effective aluminum halohydrate salts, known as activated aluminum chlorohydrates, are described in, for example, EP 0,006,739 (Gosling, et al., Unilever NV).

Antiperspirant activities containing zirconium may be represented by the empirical general formula: ZrO(OH)_{2n-nz}B_{z}·wH₂O in which z is a variable in the range of from 0.9 to 2.0 so that the value 2n-nz is zero or positive, n is the valency of B, and B is selected from the group consisting of chloride, other halide, sulphamate, sulphate and mixtures thereof. Possible hydration to a variable extent is represented by wH₂O. B may represent chloride and the variable z may lie in the range from 1.5 to 1.87. In practice, such zirconium salts are usually not employed by themselves, but as a component of a combined aluminum and zirconium-based antiperspirant with Al:Zr ratio of 10:1 to 1:10 and Metals: Cl of 0.9 to 2.1.

The above aluminum and zirconium salts may have coordinated and/or bound water in various quantities and/or may be present as polymeric species, mixtures or complexes. In particular, zirconium hydroxy salts often represent a range of salts having various amounts of the hydroxy group. Zirconium aluminum chlorohydrate may be particularly effective.

Antiperspirant activities based on the above-mentioned astringent aluminum and/or zirconium salts may be employed. The complex may include a compound with a carboxylate group such as amino acids. Examples of suitable amino acids include dl-tryptophan, dl-β-phenylalanine, dl-valine, dl-methionine and β-alanine, and glycine which has the formula CH₂(NH₂)COOH. These complexes may also include Aluminum chlorohydrex PG, Aluminum chlorohydrex PEG. PG—Propylene Glycol, PEG—Polyethylene Glycol. Other polyhydric alcohols can also be used in place of PG or PEG.

It is also desirable to employ antiperspirant active complexes of a combination of aluminum halohydrates and zirconium chlorohydrates together with amino acids such as glycine, which are disclosed, for example, in U.S. Pat. No. 3,792,068 (Luedders et al) herein incorporated by reference. Certain of those Al/Zr complexes are referred to as “ZAG” in the literature. ZAG actives may contain aluminum, zirconium and chloride with an Al/Zr ratio in a range from 2 to 10, especially 2 to 6, an Al/Cl ratio from 2.1 to 0.9 and a variable amount of glycine. Actives of this type are commercially available from many sources.

The proportion of solid antiperspirant salt in a suspension composition may include the weight of any water of hydration and any complexing agent that also may be present in the solid active.

Deodorant Actives

Suitable deodorant actives may include effective concentrations of antiperspirant metal salts, deoperfumes, and/or microbicides, including for example, bactericides, such as chlorinated aromatics, including biguanide derivatives, such as Igasan DP300™ (triclosan), Tricloban™, and Chlorhexidine. In addition, biguanide salt actives such as those are available under the trade mark Cosmocil™ may be used. Deodorant actives may be employed at a concentration of from about 0.1 to about 25% by weight. Additional deodorant actives include but are not limited to Ethylhexylglycerin, Caprylic Acid, Polyglycerol Caprylate, Xylitol, Phenoxyethanol, and 1,2-hexanediol caprylyl glycol

Optional Ingredients

Cosmetic compositions of the present invention may contain optional components. Optional components may include, for example, colorants, perfumes/fragrances, thickeners, particulate and filler materials, distributing agents, emulsifiers, wash-off agents, bacteriostats, fungistats, and mixtures thereof. Optional components useful herein are described in, for example, Geria, “Formulation of Stick Antiperspirants and Deodorants”, Cosmetics and Toiletries, 99:55-68 (1984) and International Cosmetic Ingredient Dictionary and Handbook by CTFA, 12th Edition (2008).

When perfumes or fragrances are used they may be present at concentrations up to around 4% and in certain embodiments from about 0.25 to about 2% by weight of the composition.

Thickeners may include wax-like materials such as beeswax, cerasin, hydrogenated castor oil, synthetic waxes such as Fisher Tropsch waxes, microcrystalline waxes, polyethylene waxes, and mixtures thereof. When thickeners are used, they may be present at concentrations up to about 5%.

Particulate and filler materials also may be included. These materials are typically used at levels from about 0.5% to about 5%, but usually less than about 3%. Suitable filler materials include colloidial silica (such as Cab-O-Sil, sold by Cabot Corp), clays (such as bentonite), hydrophobic (quaternized) clays, silica/alumina thickeners, silicate powders such as talc, alumina silicate, and magnesium silicate, modified corn starches, metallic stearates, and mixtures thereof. Non-limiting examples of other particulate materials include particulate hydrophilic polymers such as cellulose ether polymers, modified starches, polyamides, and polypeptides.

Emulsifiers may include non-ionic surfactants useful for forming water-in-oil emulsions. The level of emulsifiers used in the present invention is typically less than about 10%, preferably less than about 5%. Non-limiting examples of emulsifiers include polyoxyethylene ethers of fatty alcohols, and polyoxyethylene-polysiloxane copolymers.

Wash-off agents may be utilized to improve the ease with which the ingredients, particularly the gelling agent and the non-polar, non-volatile oils, may be washed off from skin or clothing. The wash-off agent is preferably a non-liquid. The wash-off agent typically comprises about 0.1% to about 10% of an antiperspirant or deodorant stick composition. Wash-off agents may include nonionic surfactants such as esters or ethers containing a C₄ to C₂₂ alkyl moiety and a hydrophilic moiety which can comprise a polyoxyalkylene group (POE or POP) and/or a polyol. Non-limiting examples wash-off agents include: ceteth-2 through ceteth-30, steareth-2 through steareth-30, ceteareth-2 through ceteareth-30, PEG-2 stearate through PEG-30 stearate, PEG-12 isostearate, PEG-16 hydrogenated castor oil, PEG-40 hydrogenated castor oil, and PEG-20 glyceryl stearate; more preferably, ceteareth-20, steareth-21, PEG-20 stearate, and PEG-16 hydrogenated castor oil; and ceteareth-20.

Additional optional components may include moisturizers, such as glycerol, in an amount up to about 5% by weight of the composition; skin benefit agents, such as allantoin or lipids, in an amount up to about 5%; skin cooling agents other than the already mentioned alcohols, such a menthol and menthol derivatives, in an amount up to about 2%.

Composition Preparation

A convenient process for preparing compositions according to the present invention may include first forming a solution of the gellant combination in the water-immiscible liquid or one of the water-immiscible liquids. This may be carried out by agitating the mixture at a temperature sufficiently high so that all gellant material dissolves (the dissolution temperature). Because of the addition of dipropylene glycol, dissolution may be carried out at a temperature in a range from about 70° C. to about 100° C. Any oil-soluble cosmetic adjunct may be introduced into an oil phase, either before or after the introduction of the gellants. The resulting solution may be allowed to cool to a temperature that is intermediate between that at which the gellants dissolved and the temperature at which it would set, often at a temperature below about 95° C. The inclusion of dipropylene glycol with a combination of linear and branched gellants such as GP-1 and EB-21 allows the pouring temperature to be lowered below about 80° C. and in some embodiments below about 75° C., 70° C., 65° C., 60° C., 55° C. and even 50° C.

In certain embodiments, it may be desirable to dissolve all or a fraction of the amide-substituted gellants in a first fraction of the composition, such as an alcohol, e.g. an alcoholic carrier fluid, i.e., a branched aliphatic alcohol, e.g. isostearyl alcohol or octyldodecanol, optionally in conjunction with an alcohol having some water-miscibility and boiling point above the dissolution temperature of DOPAD in the alcoholic fluid. This may allow the remainder of the carrier fluids to avoid being heated to temperatures at which the gellants dissolve or melt. The proportion of the carrier fluids for dissolving the gellants may be from about 15 to about 65% by weight of the carrier fluids, and in some embodiments from about 20 to about 40%.

Particulate material may be introduced into a second fraction of the carrier liquids, for example silicone and/or ester and/or hydrocarbon oils, and thereafter a first fraction containing dissolved gellants and a second fraction containing suspended particulate material are mixed at a temperature above that at which the composition gels, and often from 5° C. to 30° C. above the regular setting temperature of the composition. Dispensing containers may then be filled and cooled. Cooling may be achieved by allowing the container and contents to cool or may be assisted by blowing ambient or refrigerated air over the containers and their contents.

Product Dispenser

Compositions according to the present invention may be housed in dispensing containers, the shape and size of which may depend on the particular materials used. An antiperspirant or deodorant stick may be housed in a barrel, commonly of circular or elliptical transverse cross section, having an open end through which the stick can pass and an opposed closed end, commonly comprising a platform or elevator that is axially moveable along the barrel. The platform may be raised by the insertion of a finger or more commonly by rotation of an externally exposed rotor wheel that rotates a threaded spindle extending axially through a co-operating threaded bore in the platform. The barrel may have a removable cap that can fit over its open end. The housing is normally made from an extrudable thermoplastic such as polypropylene or polyethylene.

### EXAMPLES

### Example 1

As shown in Table 1 the combination of dipropylene glycol, GP-1 and EB-21 results in lower pour temperatures of cosmetic antiperspirant and deodorant compositions when compared with compositions which use other polyhydric alcohols, such as propylene glycol and butylene glycol.

TABLE 1

| | A | B | C | D |
|---|---|---|---|---|
| CTFA Name | % | % | % | % |
| Cyclopentasiloxane | 44.00 | 54.00 | 44.00 | 44.00 |
| Butylene Glycol | 0.00 | 0.00 | 0.00 | 10.00 |
| Propylene Glycol | 0.00 | 0.00 | 10.00 | 0.00 |
| Dipropylene Glycol | 10.00 | 0.00 | 0.00 | 0.00 |
| Octyldodecanol | 16.00 | 16.00 | 16.00 | 16.00 |
| GP-1 | 2.00 | 2.00 | 2.00 | 2.00 |
| EB-21 | 2.00 | 2.00 | 2.00 | 2.00 |
| Al/Zr Octachlorohydrex | 26.00 | 26.00 | 26.00 | 26.00 |
| Total | 100.00 | 100.00 | 100.00 | 100.00 |
| Pour Temperature | 50° C. | 95° C.† | 120° C.† | 120° C.† |

†Set up too fast to be poured.

### Example 2

As shown in Table 2, the combination of dipropylene glycol, GP-1 and EB-21 results in lower pour temperatures of cosmetic antiperspirant and deodorant compositions when compared with compositions which do not include these three components.

| | A | B | C | D | E |
|---|---|---|---|---|---|
| CTFA Name | % | % | % | % | % |
| Cyclopentasiloxane | 30.00 | 30.00 | 30.00 | 40.00 | 40.00 |
| Dipropylene Glycol | 10.00 | 10.00 | 10.00 | 0.00 | 0.00 |
| Octyldodecanol | 20.00 | 20.00 | 20.00 | 20.00 | 20.00 |
| GP-1 | 6.00 | 7.50 | 0.50 | 6.00 | 0.50 |
| EB-21 | 2.00 | 0.50 | 7.50 | 2.00 | 7.50 |
| Talc | 6.00 | 6.00 | 6.00 | 6.00 | 6.00 |
| Al/Zr | 26.00 | 26.00 | 26.00 | 26.00 | 26.00 |
| Octachlorohydrex | | | | | |
| Total | 100.00 | 100.00 | 100.00 | 100.00 | 100.00 |
| Pour Temperature | 65-67° C. | 74° C. | 78° C. | 90° C. | 95° C. |

As shown in Table 3 and 4, the combination of dipropylene glycol, GP-1 and EB-21 also results in lower pour temperatures of cosmetic sunscreen compositions.

TABLE 3

| CTFA Name | % |
|---|---|
| Octinoxate | 8.00 |
| Octisalate | 5.00 |
| Avobenzene | 2.00 |
| Dipropylene Glycol | 10.00 |
| Cyclopentasiloxane | 55.00 |
| Octyldodecanol | 16.00 |
| GP-1 | 2.00 |
| EB-21 | 2.00 |
| Total | 100.00 |

TABLE 4

| CTFA Name | % |
|---|---|
| Octinoxate | 8.00 |
| Octisalate | 5.00 |
| Avobenzene | 2.00 |
| Oxybenzone | 4.00 |
| Homosalate | 5.00 |
| Dipropylene Glycol | 10.00 |
| Cyclopentasiloxane | 39.50 |
| Octyldodecanol | 16.00 |
| Cetearyl ethoxyhexanoate | 1.00 |
| Methylparaben | 0.25 |
| Propylparaben | 0.10 |
| cyclomethicone, dimethicone, | 4.00 |
| phenyltrimethicone | |
| Phenoxyethanol | 1.00 |
| Ethylparaben | 0.15 |
| GP-1 | 2.00 |
| EB-21 | 2.00 |
| Total | 100.00 |

What is claimed is:

1. A cosmetic antiperspirant or deodorant composition comprising a branched N-acyl substituted amino acid amide gellant; a linear N-acyl substituted amino acid amide gellant; and dipropylene glycol.

2. The cosmetic composition of claim 1, wherein the branched N-acyl substituted amino acid amide gellant is EB-21.

3. The cosmetic composition of claim 1, wherein the linear N-acyl substituted amino acid amide gellant is GP-1.

4. The cosmetic composition of claim 1, wherein the branched N-acyl substituted amino acid amide gellant is EB-21 and the linear N-acyl substituted amino acid amide gellant is GP-1.

5. The cosmetic composition of claim 4, wherein the composition further comprises antiperspirant and/or deodorant actives.

6. The cosmetic composition of claim 5, wherein the composition further comprises antiperspirant actives selected from the group comprising aluminum halohydrates, zirconium chlorohydrates, or a mixture thereof.

7. The cosmetic composition of claim 5, wherein the composition further comprises one or more carrier liquids.

8. The cosmetic composition of claim 1, wherein the composition further comprises one or more optional ingredients

9. The cosmetic composition of claim 5, wherein the composition further comprises one or more aliphatic alcohols.

10. The cosmetic composition of claim 5, wherein the composition further comprises octyldodecanol.

11. The cosmetic composition of claim 5, wherein the composition further comprises one or more fragrances.

12. The cosmetic composition of claim 5, wherein the composition further comprises one or more thickeners.

13. The cosmetic composition of claim 5, wherein the composition further comprises one or more particulate and filler materials, selected from the group comprising colloidial silica, clays, hydrophobic clays, silica thickeners, alumina thickeners, silicate powders such as talc, alumina silicate and magnesium silicate, modified corn starches, metallic stearates, particulate hydrophilic polymers such as cellulose ether polymers, polyamides and polypeptides, and mixtures thereof.

14. The cosmetic composition of claim 5, wherein the composition further comprises one or more distributing agents.

15. The cosmetic composition of claim 5, wherein the composition further comprises one or more emulsifiers.

16. The cosmetic composition of claim 5, wherein the composition further comprises one or more wash-off agents.

17. The cosmetic composition of claim 5, wherein the pouring temperature of the cosmetic composition is below about 80° C.

18. The cosmetic composition of claim 5, wherein the pouring temperature of the composition is 70° C.

19. The cosmetic composition of claims 5, wherein the pouring temperature of the composition is below about 60° C.

20. The cosmetic composition of claims 1, wherein the dipropylene glycol is found in the composition at a concentration of about 0.1% about 40%.
